(19) United States
(12) Patent Application Publication
(10) Pub. No.: US 2021/0379294 A1
(43) Pub. Date: Dec. 9, 2021

(54) Needle Cover for a Medical Injection Device

(71) Applicant: Becton Dickinson France, Le Pont de Claix (FR)

(72) Inventors: Gaëlle Pouget, Grenoble (FR); Senturk Ozturk, Echirolles (FR)

(21) Appl. No.: 17/288,147

(22) PCT Filed: Oct. 21, 2019
(86) PCT No.: PCT/EP2019/078585

§ 371 Date: Apr. 23, 2021

(30) Foreign Application Priority Data
Oct. 26, 2018 (EP) 18306403.9

(51) Int. Cl. A61M 5/32

(52) U.S. Cl. CPC A61M 5/3202; A61M 2005/3109

(57) ABSTRACT

The disclosure relates to a needle cover for protecting a needle mounted on a tip of a medical injection device. The tip comprises a distal bulge. The needle cover comprises an inner needle shield made of a material with elastomeric properties. The inner needle shield comprises an inner sealing portion configured to sealingly contact the outer surface of the bulge, wherein the inner sealing portion comprises one or more ribs extending inwardly along the circumference of the inner sealing portion. At least one rib is a continuous rib in the form of a ring configured to provide a continuous contact with the outer surface of the bulge.

### CROSS-REFERENCE TO RELATED APPLICATIONS

This application is the United States national phase of International Application No. PCT/EP2019/078585 filed Oct. 21, 2019, and claims priority to European Patent Application No. 18306403.9 filed Oct. 26, 2018, the disclosures of which are hereby incorporated by reference in their entirety.

### BACKGROUND OF THE INVENTION

### Field of the Invention

The disclosure relates to a needle cover adapted to be mounted on a tip of a medical injection device for covering a needle attached thereon. The disclosure also relates to a medical assembly for use in delivering a medical composition to a body of a patient, comprising a medical injection device and a needle cover for enclosing the needle of the medical injection device.

### Description of Related Art

Medical injection devices such as syringes typically include a container for containing a medical composition having an end piece in a form of a longitudinal tip defining a fluid path through which the medical solution is expelled from the container and/or reservoir. A needle is attached to the tip in order to prick the patient's skin and perform the injection of the composition.

In order to prevent any injury prior to final use, a needle cover is mounted on the tip so as to enclose the needle. This renders the needle physically inaccessible by the persons around the device. The needle cover comprises an inner needle shield, in a material with elastomeric properties, and may further comprise an outer needle shield, in rigid plastic, surrounding the inner needle shield.

The inner needle shield ensures the sealing of the medical injection device. To that purpose, the inner needle shield comprises a sealing portion that sealingly contacts the outer surface of the bulge of the syringe's tip to provide a tight seal. The inner needle shield prevents any contamination of the medical composition from the outside environment, thereby assuring the container closure integrity. The inner needle shield further prevents any leakage of composition from the outlet of the needle to the external environment. To that purpose, the needle is preferably pricked in the inner needle shield.

A drawback of the known needle covers is that it may be relatively difficult to remove them from the tip. In order to do so, the user has to grip both the injection device and the needle cover, and to pull the needle cover by exerting an effort which may be quite important.

The force needed to remove a needle cover is measured by a physical parameter called “pull out force” (acronym POF). The pull out force necessary for removing the known needle covers from an injection device, such as a syringe, may be quite high.

As a consequence, a user having a reduced strength, for example weakened by a disease, may not be able to remove the needle shield and use the injection device for his treatment.

### SUMMARY OF THE INVENTION

The disclosure aims to provide a needle cover for a medical injection device that allows reducing the pull out force while still providing a tight sealing with the tip of the medical injection device.

To this end, one object of the disclosure is a needle cover for protecting a needle mounted on a tip of a medical injection device, wherein the tip comprises a distal bulge, the needle cover comprising an inner needle shield made of a material with elastomeric properties, the inner needle shield comprising an inner sealing portion configured to sealingly contact the outer surface of the bulge, the inner sealing portion comprising one or more ribs extending inwardly along the circumference of the inner sealing portion, at least one rib being a continuous rib in the form of a ring configured to provide a continuous contact with the outer surface of the bulge.

The one or more ribs of the inner sealing portion causes a global reduction of the pull out force, while maintaining the container closure integrity and the sealing performances of the inner needle shield with respect to the tip of the medical injection device.

According to other optional features of the needle cover:
the sealing portion comprises one, two, or three ribs, preferably one or two ribs, and more preferably two ribs. Having three ribs or less leads to a significant reduction of the pull out force compared to having four ribs or more and compared to having no rib. Having one or two ribs leads to the greatest reduction of the pull out force. Having two ribs leads to a significant reduction of the pull out force while having two sealing barriers;
each rib has preferably a rounded shape. The rounded top of the rib flattens against the outer surface of the tip's bulge, thereby improving the pressure distribution over the surface contact area between the rib and the bulge, thereby further reducing the pull out force;
the height of each rib is between 0.1 mm and 0.4 mm. The height of a rib is the distance between the base of the rib and the top of the rib intended to contact the outer surface of the bulge of the tip. Such range, defining small ribs, leads to minimal values of the pull out force compared to higher ribs;
each rib extends inwardly in an orthogonal direction relative to a longitudinal axis of the needle shield;
each rib is in the form of a continuous ring configured to provide a continuous contact with the outer surface of the bulge;
the inner needle is made of one of the following materials with elastomeric properties: a thermoplastic elastomer, an elastomer, a rubber;
when the sealing portion comprises two or three ribs, the distance between adjacent ribs is preferably comprised between 0.4 mm and 2.8 mm and more preferably between 0.4 mm and 1.2 mm;
at least one rib is in the form of a discontinuous ring configured to provide a discontinuous contact with the outer surface of the bulge;
the continuous rib is rotationally symmetrical with respect to an axis of the needle cover;
the needle cover may comprise only the inner needle shield or it may further comprise an outer needle shield surrounding at least partially the inner needle shield. The outer needle shield is preferably in rigid plastic;

Another object is a medical assembly comprising:
a medical injection device comprising:
   a main body defining a container for containing a medical composition,
   a tip extending distally from the main body, defining a fluid path extending through the tip and in fluid communication with the container, wherein said tip comprises a distal bulge,
   a needle attached to the tip and in fluid communication with the fluid path,
a needle cover as described previously, mounted on the tip of the medical injection device, wherein one or more ribs of the sealing portion of the inner needle shield sealingly contact the outer surface of the bulge.

According to other optional features of the medical assembly:
the tip further comprises a proximal cylindrical part located proximally from the distal bulge, the distal bulge having a greater diameter than the proximal cylindrical part;
the inner sealing portion contacts the outer surface of the bulge;
the tip of the medical injection device is made of glass.

### BRIEF DESCRIPTION OF THE DRAWINGS

Further features and advantages of the disclosure will become apparent from the detailed description to follow, with reference to the appended drawings, in which:

FIGS. 1A and 1B are general views of an embodiment of an injection device, respectively without a needle cover and with a needle cover attached to the tip of the injection device;

FIG. 2 is a sectional view of an embodiment of a needle cover;

FIG. 3 is a sectional view of a medical assembly comprising the medical injection device and the needle cover of FIG. 2, wherein the needle cover is attached to the tip of the injection device so as to cover the needle. The needle has not been represented on the FIG.;

FIG. 4 illustrates several designs of needle shields obtained by molding, referenced M1, M2, M3.

FIGS. 5A to 5C are views by tomography of the designs of FIG. 4 assembled in a medical injection device, wherein FIG. 5A corresponds to design M3, FIG. 5B corresponds to design M2, FIG. 5C corresponds to design M1;

FIG. 6 is a graph illustrating the value of the pull out force for removing, from the syringe's tip, the needle shields represented on FIGS. 5A to 5C;

FIG. 7 is a graph illustrating the pressure decay when air is injected in a syringe having a needle covered by the needle shields represented on FIGS. 5A to 5C;

FIGS. 8A to 8K illustrate different designs for the sealing portion of the needle shield;

FIG. 9 represents an example of results obtained when the force needed to remove a needle cover is recorded as a function of the displacement of the needle cover.

### DETAILED DESCRIPTION OF THE INVENTION

The disclosure proposes a needle cover, comprising an inner needle shield, configured to be attached to the tip of a medical injection device provided with a needle, so as to protect the needle.

When the needle cover is attached to the injection device, the combination of the needle cover and the injection device forms a medical assembly that prevents a user from contacting the needle enclosed in the needle cover, while protecting the needle from any external contamination.

The medical injection device is preferably a syringe.

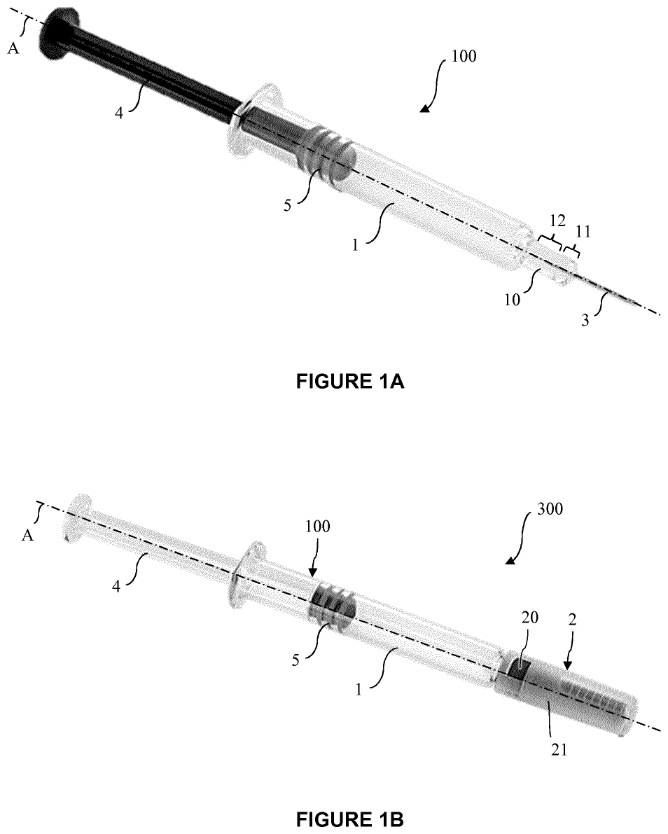

As illustrated in FIG. 1A, the medical injection device 100 comprises a body 1 extending along a longitudinal axis A, adapted to contain a medical composition to be injected, and a plunger rod 4 provided at its distal end with a stopper 5. The plunger rod 4 is configured to move translationally inside the body from a proximal position to a distal position for injecting the composition.

The medical injection device 100 further comprises a distal tip 10 extending along the axis A from the distal end of the body 1. The distal tip 10 is partially hollow so as to form a channel in fluidic communication with the body.

A needle 3 is attached to the tip 10 of the injection device.

When the plunger rod 4 is actuated and moves from the proximal position to the distal position, the stopper 5 pushes the composition from the body 1 to the tip 10 wherein said composition flows through the needle 3 and is then expelled from the injection device.

The medical injection device is preferably made of glass, and more preferably is a glass syringe. Such glass syringes are largely used in hospital environment and readily sterilizable. The medical injection device is preferably a prefilled syringe. The medical injection device is more preferably a syringe with a staked needle.

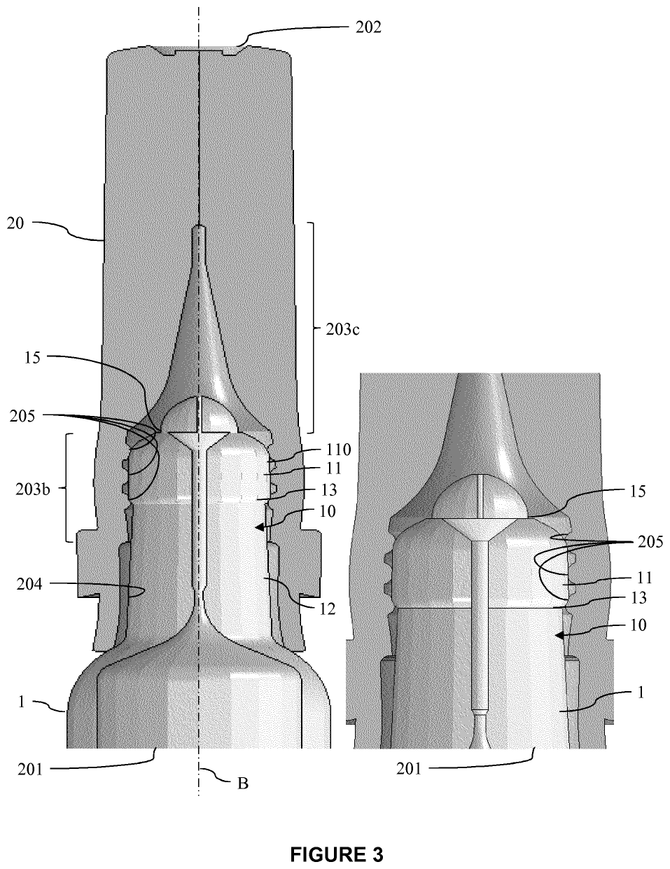

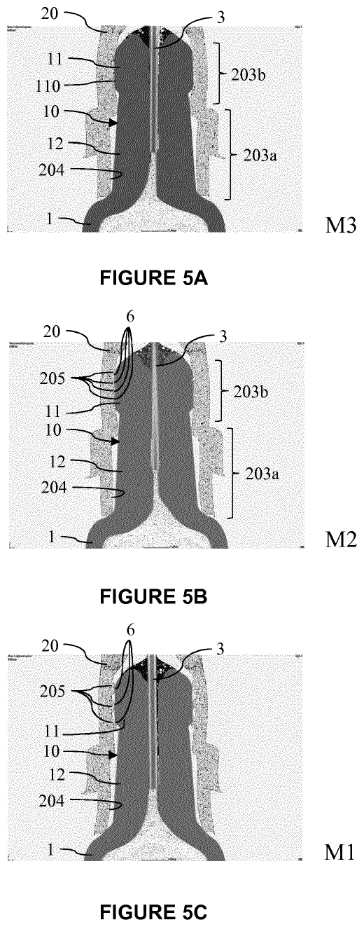

In a known manner, the tip 10 of the injection device comprises a proximal cylindrical part 12 and a distal bulge 11 located distally from the proximal cylindrical part 12. Such a bulge is illustrated in FIG. 3 and in FIGS. 5A to 5C.

A bulge 11 is a radial extension of the cylindrical part 12, of a general rounded shape, with a substantially circular section. The bulge 11 protrudes radially from the proximal cylindrical part 12 of the tip, and comprises an outer surface 110.

The bulge 11 is separated from the proximal cylindrical part 12 by a shoulder 13 that operates a diameter change along the tip 10, thereby delimitating the bulge 11 from the proximal cylindrical part 12 of the tip.

The bulge is located at the distal end of the tip.

The needle cover 2 further comprises an inner needle shield 20. The inner needle shield is made of a material having elastomeric properties, such as thermoplastic elastomer (TPE), elastomer, or rubber. Materials with elastomeric properties that are sterilizable are preferred.

As compared to plastic syringes which are injection molded, the external dimensions of a glass syringe—in particular of the tip—are less precisely controlled, due to the manufacturing process of said syringes.

Materials with elastomeric properties are particularly suited for sealing glass tips, because they comply with the external shape of the tip. Such a sealing could hardly be obtained with a rigid plastic material. Rigid inner shields are rather used for sealing plastic tips with controlled external dimensions.

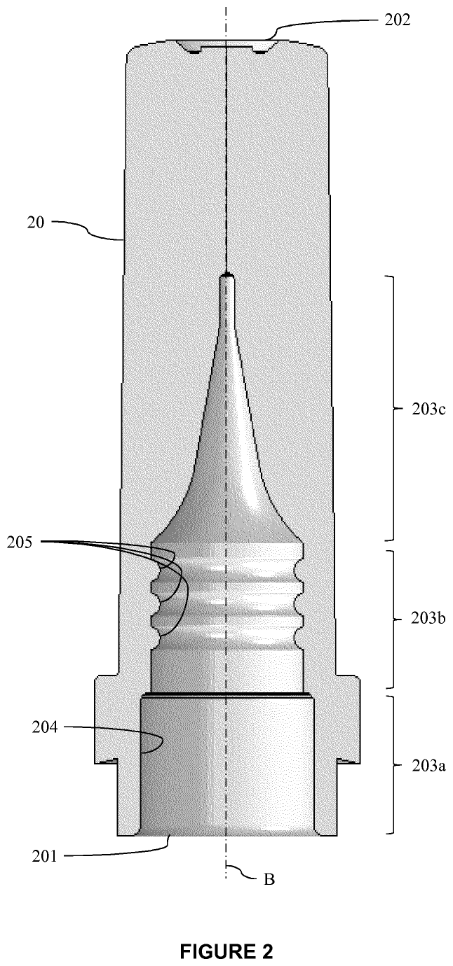

The needle shield 20 is configured to be mounted on the tip 10 of the medical injection device 100, such that an inner sealing portion 203b of the inner needle shield contacts the outer surface 110 of the bulge. FIG. 1B illustrates the syringe of FIG. 1A with a needle cover 2 attached to the tip of the syringe, thus forming a medical assembly 300. FIGS. 2 and 3 also illustrate an embodiment of the needle cover 2, respectively separated from the injection device 100 and attached to the tip 10 of the injection device 100.

More precisely, the inner sealing portion 203b of the inner needle shield 20 is configured to tightly and sealingly contact the outer surface 110 of the bulge 11. The inner sealing portion thus fulfills two sealing functions: preventing a medical composition contained in the medical injection device from leaking to the outside, and preventing external contaminants from entering into the medical injection device to maintain its integrity.

According to the disclosure, the inner sealing portion 203b of the inner needle shield 20 comprises one or more ribs 205 extending inwardly along the circumference of the inner sealing portion 203b. In other terms, the ribs 205 extend radially from the inner surface 204 of the inner sealing portion 203b to the outer surface 110 of the bulge 11 so as to sealingly contact said bulge.

Hence, a contact surface area is formed between the ribs 205 and the outer surface 110 of the bulge 11. Each contact surface area forms substantially a circle, for each rib, that extends around the bulge, of a continuous manner or a discontinuous manner.

A “continuous rib” is a rib that extends continuously along the circumference of the housing, with no interruption, so as to form a ring. A continuous rib thereby provides a continuous contact with the outer surface of the bulge.

On the contrary, a “discontinuous rib” is a rib that extends discontinuously along the circumference of the inner sealing portion, with one or more interruption, so as to form an opened ring or at least two separate parts of a ring. A discontinuous rib thereby provides a discontinuous contact with the outer surface of the bulge.

At least one rib 205 is a continuous rib configured to provide a continuous contact with the outer surface 110 of the bulge 11. When present, the other ribs 205 may be continuous or discontinuous. The presence of at least one continuous rib ensures container closure integrity while avoiding any path for leakage.

The continuous rib is preferably rotationally symmetrical with respect to an axis B of the needle cover (which coincides with the axis A of the injection device). In other terms, the continuous rib is symmetrical at each of its points relative to the axis of the needle cover. The bulge is also rotationally symmetrical with respect to an axis of the tip, which coincides with the axis B of the needle cover.

The one or more ribs 205 of the inner sealing portion 203 reduce the surface contact area between the needle cover 2 and the tip 10 of the injection device, and modify the contact pressure profile along this surface contact. Moreover, the ribs 205 may increase locally the contact pressure at such surface contact area. Surprisingly, this causes a global reduction of the pull out force, while maintaining the container closure integrity and the sealing performances of the needle cover with respect to the tip of the medical injection device.

This result is unexpected, especially since the reduction of the surface contact between the inner sealing portion 203b and the bulge 11, resulting from the presence of the ribs, would be generally related to a loss of the sealing performances. Contrary to the disclosure, conventional needle shields made of materials with elastomeric properties usually present smooth sealing portions.

The needle cover 2 may further comprise an outer needle shield 21 surrounding at least partially the inner needle shield 20 so as to enclose and protect said inner needle shield. To that end, the outer needle shield 21 is preferably made of a rigid material. According to a preferred embodiment, the outer needle shield 21 is in a rigid plastic.

According to the embodiment illustrated in FIGS. 2 and 3, the inner needle shield 20 comprises a closed distal end 202 and an opened proximal end 201.

The inner needle shield 20 further comprises an inner surface 204. The inner surface 204 preferably has a circular cross section.

The inner needle shield 20 comprises a plurality of portions of different sections:
a first portion 203a, that extends from the opened proximal end 201 up to a more distal region of the inner needle shield 20. The first portion 203a has preferably a greater diameter than the other portions of the inner needle shield,
a second portion 203b, which is the inner sealing portion detailed previously, preferably having a reduced section compared to the first section 203a. The inner sealing portion extends from the first portion 203a up to a more distal region of the inner needle shield, and
a third portion 203c, that preferably tapers from the second portion 203b to the distal end of the inner needle shield 20.

The first portion 203a is configured to accommodate the proximal part 12 of the tip; the second portion 203b, which is the inner sealing portion, is configured to contact the bulge 11 in a sealing way; and the third portion 203c is configured to accommodate the needle 3. More precisely, the third portion 203c is configured so that the needle 3 may be pricked into a distal part of this third portion.

The inner sealing portion 203b comprises at least one rib 205, three ribs being illustrated in the needle shield of FIGS. 2 and 3, which extend(s) radially inwardly along the circumference of the inner sealing portion.

When the inner sealing portion 203b comprises several ribs, the ribs 205 are preferably parallel to each other.

The three ribs of FIGS. 2 and 3 are continuous ribs configured to provide a continuous contact with the outer surface of the bulge. However, it has to be known that only one or two of the three ribs may be continuous.

At least one rib 205 is configured to sealingly contact the outer surface of the bulge. In a preferred embodiment, two ribs 205 are configured to sealingly contact the outer surface 110 of the bulge 11. In the represented embodiment, the three ribs are configured to sealingly contact the outer surface 110 of the bulge 11. As illustrated in FIG. 3, when the needle cover 2 is attached to the tip 10 of the injection device, the bulge 11 is located in the second portion 203b of the housing, and the top of the ribs 205 contacts the outer surface 110 of the bulge.

The ribs 205 are configured to exert a radial pressure onto the bulge 11. The total pressure exerted by the ribs onto the bulge may be controlled by adjusting several parameters, among which the section of the inner sealing portion 203b relative to that of the bulge 11, the dimensions of each rib 205, the number of ribs, and the distance between adjacent ribs. Some of these parameters will be described in more details in the following, in view of tests presented in the examples.

The ribs 205 are preferably formed in one piece with the inner needle shield 20, and are advantageously made of the same material as the inner needle shield.

According to a preferred embodiment, the ribs 205 have a rounded shape, namely, their top is curved and points inwardly towards the longitudinal axis of the needle cover. As such, when contacting the bulge 11, the rounded top of the rib flattens against the outer surface of the tip's bulge, thereby improving the pressure distribution over the surface contact area between the rib 205 and the bulge 11, thereby further reducing the pull out force. Such a flattening is allowed by the elastomeric properties of the inner shield and would not be obtained with a rigid material.

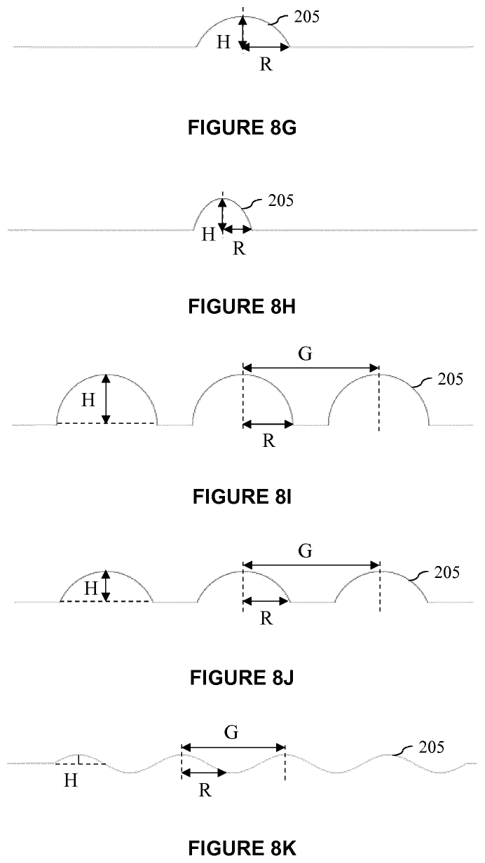

According to an embodiment, the distance between adjacent ribs is comprised between 0.4 mm and 2.8 mm, preferably 0.4 mm and 1.2 mm. This range of distances provides the most important diminution of the pull out force. The distance is the gap between the top of the ribs, noted “G” in FIGS. 8A and 8E.

The distance between two adjacent ribs, as every other dimension of the inner needle shield, is measured when the inner needle shield is not mounted on the syringe's tip. As a matter of fact, once the inner needle shield is mounted on the syringe's tip, the ribs flattens against the bulge, such that the dimensions of the ribs may vary compared to their dimensions when the inner needle shield is not inserted onto the syringe's tip.

According to an embodiment, the height H of each rib, which is the distance between the base of a rib and the top of the rib, is between 0.1 mm and 0.4 mm. This range of height provides the most important diminution of the pull out force.

According to an embodiment, the radius R of each rib, at the base of a rib, is between 0.1 mm and 0.4 mm in order to provide the most important diminution of the pull out force.

While the inner needle shield of the disclosure has been described with reference to the FIGS., the disclosure is not limited to the embodiments represented on the FIGS. For example, the needle cover could have a different shape than the shape described with reference to the FIGS. Besides, the first portion 203a and/or the third portion 203c of the inner needle shield may have different shapes than those represented on the FIGS.

For example, according to some embodiments, the first portion 203a of the inner needle shield 20 may further comprise an anti-pop off rib, as disclosed in document EP1208861, for preventing the needle shield from coming off from the tip of the injection device during sterilization.

When present, the anti-pop off rib extends inwardly along the circumference of the inner surface 204 of the first portion 203a of the inner needle shield 20 which is intended to come into contact with the proximal cylindrical part 12 of the tip 10. The anti-pop off rib is then located proximally relative to the inner sealing portion 203b and the shoulder 13, and configured to contact the proximal cylindrical part 12.

More precisely, the anti-pop off rib is configured to abut the shoulder 13 when the needle cover 2 moves in a distal direction relative to the tip 10 of the medical injection device, thereby preventing the inner needle shield 20 from disengaging the tip 10.

An embodiment of the anti-pop off rib is disclosed in document EP1208861. In this document the anti-pop off rib is positioned proximally from the shoulder between the bulge and the main portion of the tip of the injection device when the needle cover is mounted on the injection device. This rib is intended to retain the needle shield on the tip during the sterilization process, wherein the pressure difference between the sterilization chamber and the housing may vary significantly.

The anti-pop off rib of document EP1208861 is not located on the inner sealing portion 203b intended to contact the bulge 11 of the tip 10. As a matter of fact, the skilled person traditionally does not modify the inner sealing portion 203b as it is intended to assure the sealing of the needle cover 2. Consequently, in the document EP1208861, the inner surface of the inner sealing portion is smooth, which reflects the general knowledge in the art of sealing, contrary to the ribbed surface of the disclosure.

### EXAMPLES: STUDIES OF VARIOUS DESIGNS OF NEEDLE SHIELDS

### Example 1

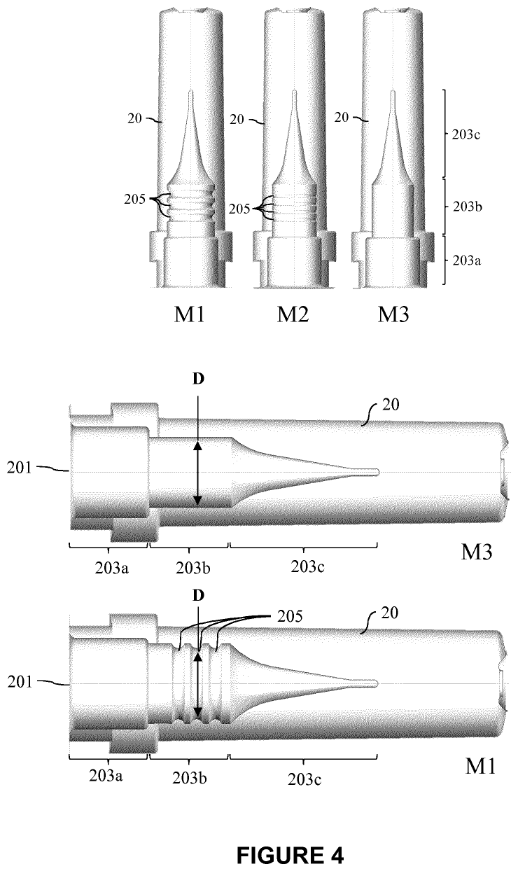

Three different designs of inner needle shields 20, noted M, are molded in thermoplastic elastomer. These inner needle shields are represented on FIG. 4. The features of these needle shields are described below and in Table 1.

TABLE 1
molded needle shields

| | Radius | Height | Gap | D |
|---|---|---|---|---|
| Design | (mm) | (mm) | (mm) | (mm) |
| M1 (large | 0.4 | 0.25 | 1.07 | 3.8 |
| ribs) | | | | |
| M2 (small | 0.2 | 0.1 | 0.71 | 3.8 |
| ribs) | | | | |
| M3 (standard | N/A | N/A | N/A | 3.8 |
| smooth) | | | | |

“Radius” (R) refers to the radius (width) of each rib; “height” (H) refers to the height of each rib; “gap” (G) refers to the distance between two adjacent ribs; and “D” refers to the internal diameter of the inner needle shield, at the inner sealing portion. For ribbed designs, the diameter is measured at the top of a rib, as illustrated in FIG. 4, when the inner needle shield is not inserted on a syringe's tip. For all the designs M1, M2, M3, the needle shield is in TPE and the injection device is a glass syringe.

The designs M1, M2, M3 illustrated in FIG. 4 will be described also in reference with FIGS. 5A to 5C that are corresponding tomography views of said designs M1, M2, M3 of FIG. 4 that have been assembled on the tip of syringes via a compression bench.

M3 (FIG. 5A) is a needle shield according to the state of the art. Such needle shield does not comprise any rib, i.e. the inner sealing portion 203b is a smooth surface.

M2 (FIG. 5B) is a needle shield according to the disclosure, comprising an inner sealing portion 203b provided with four small ribs 205. The surface contact area between the inner sealing portion 203b and the bulge 11 is defined by the plurality of small contact zones 6 between the ribs 205 and the bulge 11. Thanks to the ribs the contact pressure is null on the non-ribbed part, or at least highly reduced on the non-ribbed part if the surface is still in contact (the pressure applied to the bulge is then negligible). This allows to decrease the pull out force.

M1 (FIG. 5C) is a needle shield according to the disclosure, comprising an inner sealing portion 203b provided with three large ribs 205. The surface contact area between the inner sealing portion 203b and the bulge 11 is defined by the plurality of small contact zones 6 between the ribs 205 and the bulge 11. This is schematically similar to that of M2 (FIG. 5B), except that the ribs are larger, with both greater radius and greater height. The surface contact area is reduced compared to the standard known design M3. Thanks to the ribs the contact pressure is null or highly reduced on the non-ribbed part, allowing to decrease the pull out force. Though, since the ribs are higher than those of M2, and because the ribs flatten less on the bulge, the surface contact area is smaller than that of M2.

The needle shields M1 and M2, provided with rib(s) on the inner sealing portion 203b, exhibit a reduced pull out force, compared to the needle shield M3 that is not provided with ribs on the inner sealing portion 203b.

1. Measurement of the Pull Out Force

Measurements of the force needed to remove the needle cover from the syringe are carried out, through 30 assays. The test is performed with a traction bench. The method comprises the steps of:
placing the syringe on a holder,
holding the needle cover with pneumatic jaws and then
pulling the needle cover at a constant displacement rate to remove it.

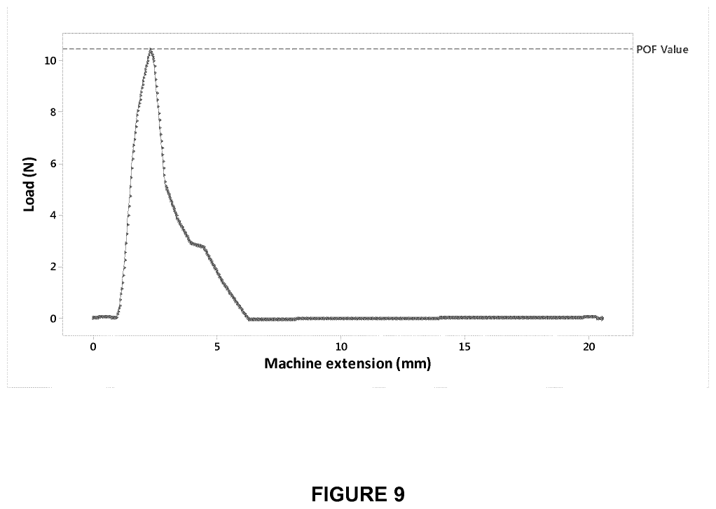

The force needed to remove the needle cover is recorded, in function of the displacement of the needle cover. As represented on FIG. 9, the force needed to remove the needle cover increases at the beginning of the movement of the needle cover, until it reaches a maximum value, named “POF value” on FIG. 9, and corresponding to the pull out force.

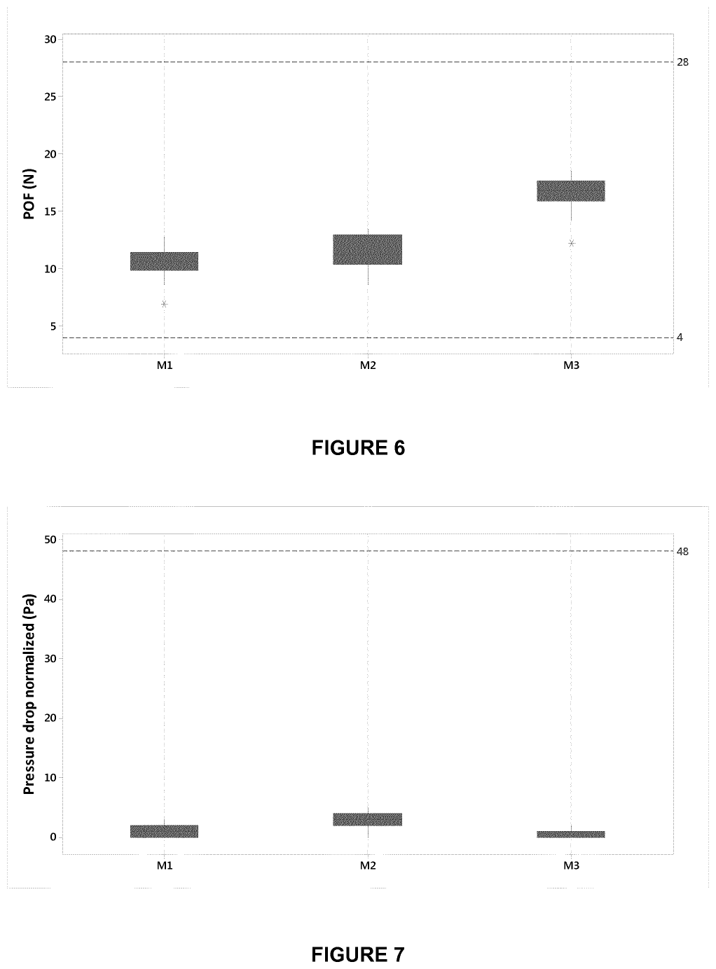

The pull out force measured by the method are indicated in Table 2 and illustrated in the graph of FIG. 6.

TABLE 2
experiments for measuring the pull out force

| | | Mean Value | | Minimum | Maximum |
|---|---|---|---|---|---|
| | | of recorded | | recorded | Recorded |
| | | force Pull | Standard | Pull out | Pull out |
| | Total | Out Force | deviation | Force | Force |
| Design | counts | (Newton) | (Newton) | (Newton) | (Newton) |
| M1 | 30 | 10.6 | 1.2 | 6.9 | 12.8 |
| M2 | 30 | 11.5 | 1.3 | 8.6 | 13.5 |
| M3 | 30 | 16.6 | 1.4 | 12.2 | 18.5 |

These results show that the presence of ribs strongly reduces the pull out force. Indeed, the pull out force of ribbed designs M1 (large ribs) and M2 (small ribs) is reduced by around 6 Newtons (40%) compared to the standard design M3 (no rib). The standard deviation StDev also tends to be lower for the ribbed designs M1 and M2 compared to the standard design M3.

The difference between large and small ribs is not significant, since the pull out force values of ribbed designs M1 (POF=10.6) and M2 (POF=11.5) are very close to each other.

The presence of ribs enables to reduce the pull out force, regardless of the geometry and size of the ribs.

2. Leak Test Pressure

15 leak assays have been carried out to assess the sealing performance of the needle shields provided with ribs on their inner sealing portion.

The leak assays have been carried out as follows: empty prefillable syringes have been capped with the needle covers M1, M2, M3 represented on FIG. 4. A pressure has been applied inside the barrel of an empty syringe for a determined period of time (1.1 bar for 5 seconds). The pressure decay in the barrel has been measured at the same time. If there is a leak at the needle cover—syringe's tip interface then a large pressure decay is measured. This test follows the pressure conditions indicated in the norm 11040-4:2015(E).

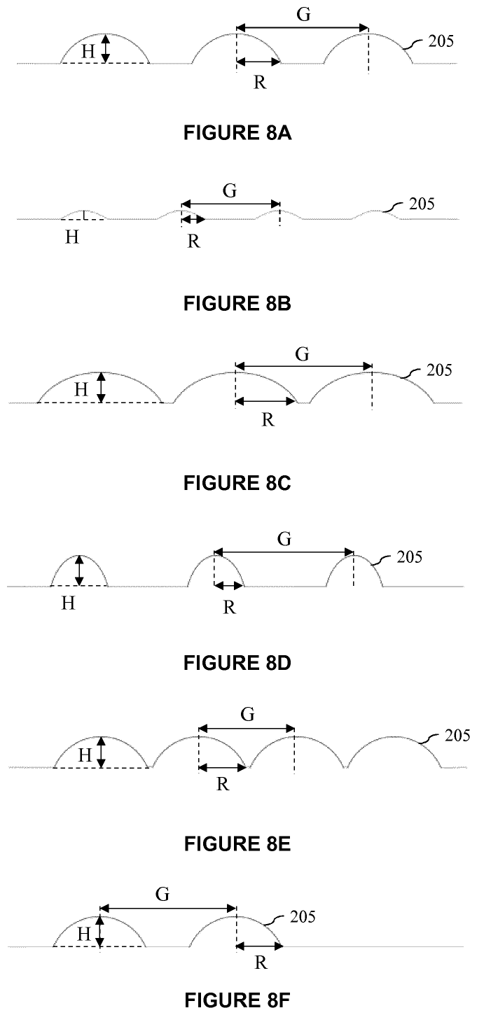

The results are illustrated in the graph of FIG. 7.

The results show that all the designs (including the known reference design M3) lead to very low pressure decay values, between 0 Pa and 2 Pa. Hence, an optimal sealing of the tip of the syringe is preserved with the presence of ribs, whatever the width and the height of the ribs. The sealing performances of the needle shield with respect to the tip of the medical injection device are maintained.

### Example 2: Development of Different Designs for the Sealing Surface of the Needle Cover

In order to study the influence of the dimensions of the ribs, as well as their number, on the pull out force, a finite element analysis has been performed. Thermoplastic elastomer material properties (similar to material molded in example 1) are used for this finite element analysis. The removal of the needle cover is simulated and the pull out force is calculated.

Simulated designs for the sealing portion of the needle cover are described below in Table 3.

TABLE 3
alternative designs for the sealing portion of the needle cover

| | | | Schematic |
|---|---|---|---|
| Trial | Design | Structural features | view |
| 1 | M1 | No change vs. M1 | FIG. 8A |
| | R = 0.4 mm | design | |
| | G = 1.07 mm | | |
| | H = 0.25 mm | | |
| 2 | M2 | No change vs. M2 | FIG. 8B |
| | R = 0.2 mm | design | |
| | G = 0.71 | | |
| | H = 0.1 mm | | |
| 3 | M1, with R = 0.55 mm | Influence of the rib's | FIG. 8C |
| | | radius (+) | |
| 4 | M1, with R = 0.2 mm | Influence of the rib's | FIG. 8D |
| | | radius (−) | |
| 5 | M1, with G = 0.8 mm | 4 ribs | FIG. 8E |
| 6 | M1, with only 2 ribs, | 2 ribs | FIG. 8F |
| | and G = 1.07 mm | | |
| 7 | M1, with only 1 rib | 1 big rib | FIG. 8G |
| 8 | M1, with only the middle | 1 small rib | FIG. 8H |
| | rib of trial 4 | | |
| 9 | M1, with H = 0.35 mm | Influence of the height | FIG. 8I |
| | | (thickness) of the rib | |
| 10 | M1, with D = 3.60 mm | Ribs with smaller inner | FIG. 8J |
| | | diameter | |
| 11 | M2, with a full sinusoid | Sinusoid shape | FIG. 8K |
| | shape | | |

Trials 1 and 2 correspond respectively to designs M1 and M2 described previously in example 1.

Trials 3 and 4 correspond to design M1, except that the radius R of each rib is 0.55 mm for trial 3 and 0.2 mm for trial 4, instead of 0.4 mm. These trials allow for assessing the effect of the width of the ribs on the pull out force (compared to design M1).

Trial 5 corresponds to design M1, except that the gap G between two adjacent ribs is 0.8 mm, instead of 1.07 mm, and the contact portion comprises 4 ribs instead of 3.

Trial 6 corresponds to design M1, except that the gap G between two adjacent ribs is 1.07 mm, instead of 0.71 mm, and the contact portion comprises 2 ribs instead of 4.

Trial 7 corresponds to design M1, except that the contact portion comprises only 1 rib instead of 4.

Both trials 6 and 7 allow for assessing the effect of the number of ribs on the pull out force (compared to design M1).

Trial 8 corresponds to design M1, except that the contact portion comprises only 1 rib instead of 4, and the one rib is the rib of trial 4 (R=0.2 mm).

Trial 9 corresponds to design M1, except that the height of each rib is 0.35 mm instead of 0.25 mm. This trial allows for assessing the effect of the height of the ribs on the pull out force (compared to design M1).

Trial 10 corresponds to design M1, except that the inner diameter of each rib is 3.60 mm instead of 3.80 mm.

Trial 11 corresponds to design M2, except that the contact portion has a full sinusoidal shape. This trial allows for assessing the pull out force when the inner surface has a sinusoid shape that does not contact the tip of the syringe.

1. Influence of the Features of the Ribs on the Pull Out Force

The calculated pull out force values for the 11 designs described above are described in Table 4.

TABLE 4
Pull out force values for the alternative designs
for the sealing surface of the needle cover

| Trial | Design | POF (Newton) |
|---|---|---|
| Standard | Standard design (no rib), | 14.9 |
| | corresponding to M3 | |
| 1 | M1 | 11.3 |
| 2 | M2 | 12.1 |
| 3 | M1, with R = 0.55 mm | 11.1 |
| 4 | M1, with R = 0.2 mm | 10.8 |
| 5 | M1, with G = 0.8 mm | 13.1 |
| 6 | M1, with only 2 ribs, and G = 1.07 mm | 7.0 |
| 7 | M1, with only 1 rib | 7.0 |
| 8 | M1, with only the middle rib of trial 4 | 6.3 |
| 9 | M1, with H = 4.15 mm | 15.3 |
| 10 | M1, with D = 3.60 mm | 10.7 |
| 11 | M2, with a full sinusoid shape | 11.5 |

a) Influence of the Width (Radius) of the Ribs on the Pull Out Force

As shown in Table 4, by comparing trials 1 and 3, when the ribs width increases from 0.4 mm for trial 1 (M1) to 0.55 mm for trial 3, the pull out force slightly decreases from 11.3 N to 11.1 N.

By comparing trials 1 and 4, when the width decreases from 0.4 mm for trial 1 (M1) to 0.2 mm for trial 4, the pull out force slightly decreases from 11.3 N to 10.8 N.

The decrease of the pull out force is very low for trials 3 and 4 and occurs both when the width is increased and decreased relative to M1.

Therefore, there is no significant influence of the width of the ribs on the pull out force.

b) Influence of the Number of Ribs on the Pull Out Force

As shown in Table 4, by comparing trials 2 and 6, when the number of ribs decreases from 4 to 2, the pull out force decreases from 12.1 N to 7.0 N.

By comparing trials 2 and 7, when the number of ribs decreases from 4 to 1, the pull out force decreases from 12.1 N to 7.0 N.

Therefore, decreasing the number of ribs causes the pull out force to decrease and conversely.

A low number of ribs, especially between 1 and 3, and in particular 1 or 2 ribs, leads to low value of pull out force and is thus preferred.

c) Influence of the Height of Ribs on the Pull Out Force

As shown in Table 4, by comparing trials 1 and 9, when the height of each rib increases from 0.25 mm to 0.35 mm, the pull out force increases from 11.3 N to 15.3 N.

Therefore, increasing the height of the ribs causes the pull out force to increase.

Ribs with low height, especially between 0.1 mm and 0.4 mm, lead to low value of pull out force and are thus preferred.

2. Influence of the Material of the Needle Shield on the Pull Out Force

In order to study the influence of the needle shield material properties, pull out forces for several materials were simulated by finite element analysis:
TPE is the material used in previous section 1.
Then three rubbers with different properties were simulated. Rubber 1 is a styrene butadiene rubber, rubber 2 a synthetic isoprene rubber, and rubber 3 a natural rubber.

Only 4 designs out of the 11 designs of Tables 3 and 4 are assessed. The pull out force values for the 4 designs and the 4 materials are described in Table 5.

TABLE 5
Pull out force values for four alternative designs for the sealing
surface of the needle cover, with four different materials

| | | | POF |
|---|---|---|---|
| Trial | Design | Material | (Newton) |
| Standard | Standard design (no rib), | TPE | 14.86 |
| | corresponding to M3 | Rubber 1 | 17.30 |
| | | Rubber 2 | 10.54 |
| | | Rubber 3 | 8.50 |
| 1 | M1 | TPE | 11.25 |
| | | Rubber 1 | 12.14 |
| | | Rubber 2 | 7.25 |
| | | Rubber 3 | 5.83 |
| 6 | M1, with only 2 ribs, and G = | TPE | 7.03 |
| | 1.07 mm | Rubber 1 | 7.07 |
| | | Rubber 2 | 3.83 |
| | | Rubber 3 | 3.07 |
| 9 | M1, with H = 4.15 mm | TPE | 15.44 |
| | | Rubber 1 | 14.50 |
| | | Rubber 2 | 9.52 |
| | | Rubber 3 | 7.70 |

As shown in Table 5, whatever the elastomeric properties of the needle shield, the results described previously in example 1 part 1 and example 2 parts 1 a), 1b), and 1c) are confirmed.

Indeed:
the pull out force is lower for the ribbed designs of trial 1 (with ribs) than for the un-ribbed standard designs (without rib), whether the material of the inner needle shield is TPE, rubber 1, rubber 2, or rubber 3;
the pull out force is lower for the designs of trial 6 with low number of ribs (M1 with only 2 ribs) than for designs of trial 1 with higher number of ribs (M1, 3 ribs), whether the material of the needle shield is TPE, rubber 1, rubber 2, or rubber 3;
the pull out force is higher for the designs of trial 9 with high ribs (M1 with H=0.35 mm) than for designs of trial 1 with lower ribs (M1, H=0.25 mm), whether the material of the needle shield is TPE, rubber 1, rubber 2, or rubber 3.

1. A needle cover for protecting a needle mounted on a tip of a medical injection device, wherein the tip comprises a distal bulge, the needle cover comprising an inner needle shield made of a material with elastomeric properties, the inner needle shield comprising an inner sealing portion configured to sealingly contact an outer surface of the bulge, wherein the inner sealing portion comprises one or more ribs extending inwardly along a circumference of the inner sealing portion, at least one rib being a continuous rib in the form of a ring configured to provide a continuous contact with the outer surface of the bulge.

2. The needle cover of claim 1, wherein the sealing portion comprises one, two, or three ribs.

3. The needle cover of claim 1, wherein each rib has a rounded shape.

4. The needle cover of claim 1, wherein the height of each rib between 0.1 mm and 0.4 mm.

5. The needle cover of claim 1, wherein each rib extends inwardly in an orthogonal direction relative to a longitudinal axis of the needle shield.

6. The needle cover of claim 1, wherein each rib is in the form of a continuous ring configured to provide a continuous contact with the outer surface of the bulge.

7. The needle cover of claim 1, wherein the material with elastomeric properties is a thermoplastic elastomer, an elastomer, or a rubber.

8. The needle cover of claim 1, wherein the inner sealing portion comprises two or three ribs, a distance between adjacent ribs being comprised between 0.4 mm and 2.8 mm, and preferably between 0.4 mm and 1.2 mm.

9. The needle cover of claim 1, wherein the continuous rib is rotationally symmetrical with respect to an axis of the needle cover.

10. The needle cover of claim 1, further comprising an outer needle shield surrounding, at least partially, the inner needle shield.

11. A medical assembly comprising:
a medical injection device comprising:
a main body defining a container for containing a medical composition,
a tip extending distally from the main body, defining a fluid path extending through the tip and in fluid communication with the container, wherein said tip comprises a distal bulge,
a needle attached to the tip and in fluid communication with the fluid path,
a needle cover as claimed in claim 1, mounted on the tip of the medical injection device,
wherein one or more ribs of the inner sealing portion of the inner needle shield sealingly contact the outer surface of the bulge.

12. The medical assembly of claim 11, wherein the tip of the medical injection device is made of glass.

13. The needle cover of claim 1, wherein the sealing portion comprises one or two ribs.

14. The needle cover of claim 1, wherein the sealing portion comprises two ribs.
